(19) United States
(12) Patent Application Publication
(10) Pub. No.: US 2009/0117053 A1
(43) Pub. Date: May 7, 2009

(54) LOCAL DELIVERY OF 5-AMINOLEVULINIC-ACID BASED COMPOUNDS TO TISSUES AND ORGANS FOR DIAGNOSTIC AND THERAPEUTIC PURPOSES

(75) Inventor: Jianmin Li, Lexington, MA (US)

Correspondence Address: MAYER & WILLIAMS PC, 251 NORTH AVENUE WEST, 2ND FLOOR, WESTFIELD, NJ, 07090, US

(73) Assignee: Boston Scientific Scimed, Inc., Maple Grove, MN (US)

(21) Appl. No.: 12/266,366

(22) Filed: Nov. 6, 2008

Related U.S. Application Data
(60) Provisional application No. 61/001,967, filed on Nov. 6, 2007.

(51) Int. Cl. A61K 49/00; A61K 31/195; A61P 35/04

(52) U.S. Cl. 424/9.61; 514/561

(57) ABSTRACT

In accordance with an aspect of the present invention, a 5-aminolevulinic-acid-based compound (ALA-based compound) is delivered locally to a tissue or organ within a subject's body. In another aspect, a method of diagnosis or treatment is provided which comprises: (a) locally delivering an ALA-based compound to a tissue or organ within a subject's body and (b) illuminating the tissue or organ with light having a wavelength suitable to induce fluorescence of protoporphyrin IX (PPIX). In some embodiments, a local therapeutic agent is delivered to the targeted tissue or organ for treatment. In another aspect, formulations containing the ALA-based compound are provided.

### STATEMENT OF RELATED APPLICATIONS

This application claims the benefit of U.S. Provisional Patent Application Ser. No. 61/001,967, filed Nov. 6, 2007, entitled “Local Delivery Of 5-Aminolevulinic-Acid Based Compounds To Tissues And Organs For Diagnostic And Therapeutic Purposes”, which is incorporated by reference herein in its entirety.

### FIELD OF THE INVENTION

This invention relates to methods in which 5-aminolevulinic acid is locally delivered to a target tissue or organ for diagnostic or therapeutic purposes.

### BACKGROUND OF THE INVENTION

Administration of 5-aminolevulinic acid (ALA) to the human body is known to result in the endogenous production of protoporphyrin IX (PP IX), a fluorescent photosensitizer, in neoplastic tissue. Under excitation with blue light, PP IX emits a characteristic visible red fluorescence. Typically, ALA is administrated either orally (see E. Malik et al., Human Reproduction, Vol. 15, No. 3, 584-588, March 2000) or infused intraperitoneally (see Martin C. Löning et al., Lasers in Surgery and Medicine, Volume 38, Issue 5, 2006, Page 549-554). These methods require 5-10 hours pre-treatment time and high dose of ALA (e.g., 30 mg/kg body weight), making them clinically inconvenient.

### SUMMARY OF THE INVENTION

In accordance with an aspect of the present invention, a 5-aminolevulinic acid-based compound (ALA-based compound) is delivered locally to a tissue or organ within a subject's body.

In accordance with another aspect of the present invention, a method is provided which comprises: (a) locally delivering an ALA-based compound to a tissue or organ within a subject's body and (b) illuminating the tissue or organ with light having a wavelength suitable to induce fluorescence of the PPIX metabolic breakdown product of the ALA-based compound. In some embodiments, a local therapeutic agent delivered to the targeted tissue or organ for treatment.

In another aspect, formulations containing die ALA-based compound are provided.

Because the ALA is administered locally, one or more of the following advantages are realized: (A) The time required for die ALA to be taken up by the tissue or organ is dramatically reduced relative to oral or intraperitoneal methods. (B) The required dose per kg of body weight is also dramatically reduced relative to oral or intraperitoneal methods. (C) Systemic distribution, and therefore side effect relating to systemic distribution, may be reduced or essentially eliminated relative to oral or intraperitoneal methods. (D) The local concentration of ALA is increased, thereby increasing the detection sensitivity of die method. (E) Diagnosis and treatment may be combined together to reduce patient discomfort and inconvenience.

These and various additional aspects, embodiments and advantages of die present invention will become immediately apparent to those of ordinary skill in the art upon review of the Detailed Description and any Claims to follow.

### BRIEF DESCRIPTION OF THE DRAWING

FIG. 1 is a schematic illustration of a delivery device, in accordance with an embodiment of the invention.

### DETAILED DESCRIPTION OF THE INVENTION

In accordance with an aspect of the present invention, a 5-aminolevulinic acid-based compound (ALA-based compound) is delivered locally to a tissue or organ within a subject's body. By locally delivering the ALA-based compound, the amount of ALA required is dramatically reduced when compared with oral or intraperitoneal administration (e.g., by ten or more times) and sensitivity and accuracy may be significantly improved.

Preferred subjects are vertebrate subjects, more preferably mammalian subjects and more preferably human subjects.

In accordance with another aspect of the present invention, a method is provided which comprises: (a) locally delivering an ALA-based compound to a tissue or organ within a subject's body and (b) illuminating the tissue or organ with light having a wavelength suitable to induce fluorescence of PPIX, the metabolic breakdown product of the ALA-based compound. Such illumination may be used to identify one or more areas having increased PPIX, which is common to neoplastic tissue such as tumors and endometrial tissue. Such illumination may also be used to photodynamically treat one or more areas having increased PPIX.

In accordance with another aspect of the present invention, a method is provided which comprises: (a) locally delivering an ALA-based compound to a tissue or organ within a subject's body, (b) illuminating the tissue or organ with light having a wavelength suitable to induce fluorescence of PPIX, and (c) delivering a therapeutic agent to the tissue or organ under direct visualization.

As used herein, the term “ALA-based compound” refers to 5-aminolevulinic acid (ALA) and its salts (5-aminolevulinic acid hydrochloride), ALA derivatives (e.g., various ALA esters and ALA thioesters) and their salts, and ALA-mimetics (i.e., compounds that result in the endogenous production of PP IX) and their salts.

As examples of ALA derivatives, US 2006/0084701 to Gierskeky et al. describes the preparation of various ALA ester hydrochlorides including ALA methyl ester hydrochloride, ALA ethyl ester hydrochloride, ALA n-propyl ester hydrochloride, ALA n-hexyl ester hydrochloride, ALA n-heptyl ester hydrochloride, and ALA n-octyl ester hydrochloride. Various ALA ester hydrochlorides including (a) alkoxyalkyl and alkoxyalkyloxy ester hydrochlorides such as

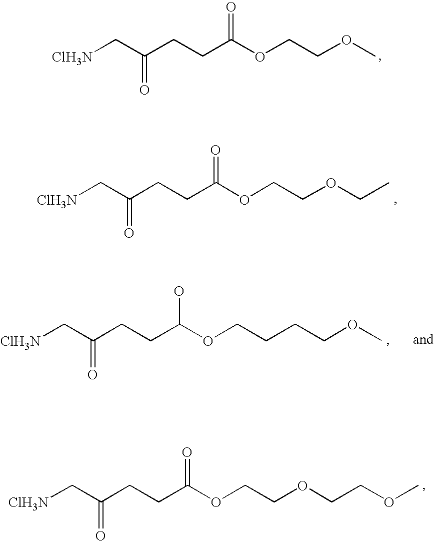

(b) ALA fluorinated alkyl ester hydrochlorides such as

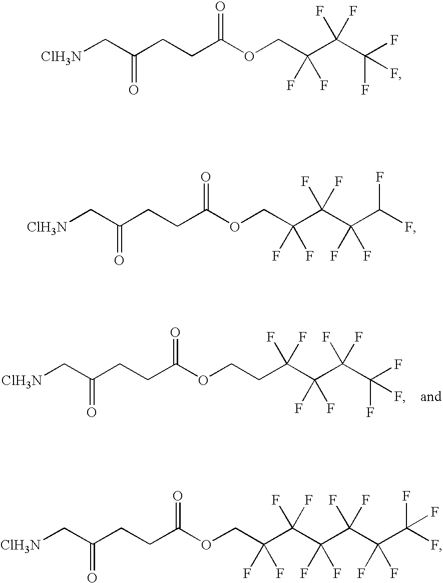

(c) AlA benzyl ester hydrochloride and its derivatives such as

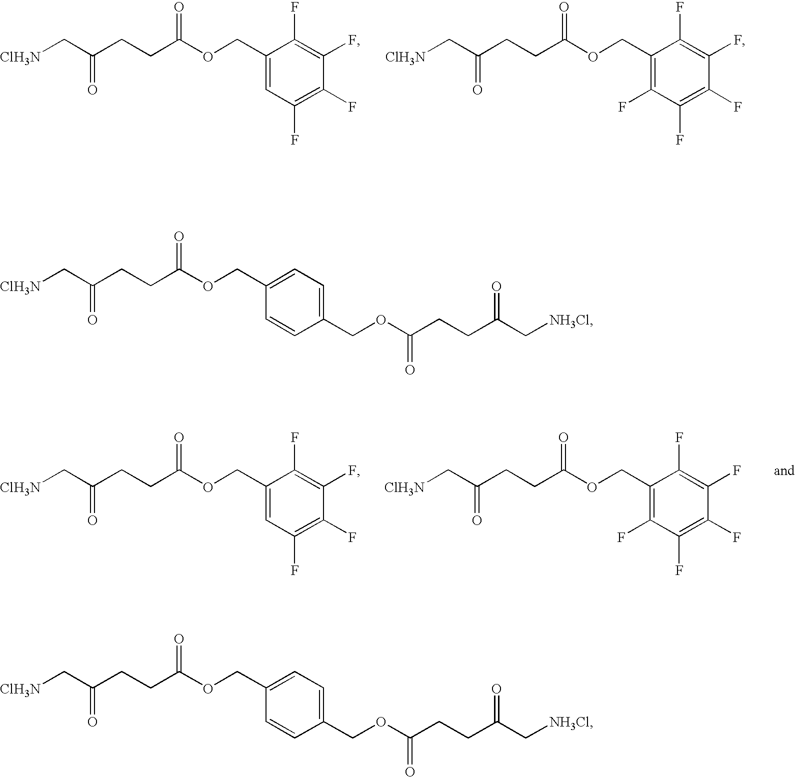

miscellaneous other ALA ester hydrochlorides such as

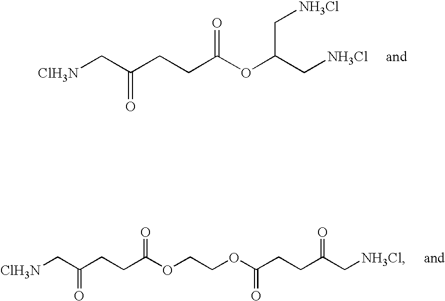

(e) ALA thiohexyl ester hydrochloride

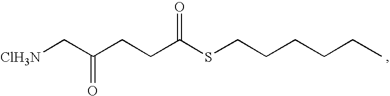

have been reported in H. Brunner et al., “New 5-aminolevulinic acid esters—efficient protoporphyrin precursors for photodetection and photodynamic therapy,” Photochem. Photobiol., November 2003, 78(5), 481-486. Brunner et al. further report that the ALA nonafluorohexylester hydrochloride, ALA thiohexylester hydrochloride and ALA dibenzyldiester dihydrochloride displayed appreciably increased PPIX levels and showed improved phototoxicity compared with ALA hydrochloride, and various other ALA ester hydrochlorides.

In another aspect of the invention, formulations containing the ALA-based compound are provided. For instance, in the methods of the present invention, the ALA-based compound is generally applied locally as a liquid formulation, for example, with the assistance of a catheter (i.e., a device comprising a hollow tube, which is inserted into the subject, and from which die liquid formulation is delivered). Typical concentrations of ALA-based compound in the liquid formulations range, for example, from 0.1 to 1 to 10 to 100 to 500 g/mL, among other possibilities.

ALA hydrochloride is soluble in water (−500 mg/mL) and thus readily administered using an aqueous vehicle. The solubility of other ALA-based compounds in various aqueous, semi-aqueous, and non-aqueous vehicles will, of course, depend upon the chemical structure of each compound. For example, ALA hydrochloride is more hydrophilic than ALA methyl ester hydrochloride, which is more hydrophilic than ALA ethyl ester hydrochloride, which is more hydrophilic than ALA n-propyl ester hydrochloride, and so forth. These differences in hydrophilicity will dictate the specific liquid vehicle that is selected, for example, whether it is based on water (i.e., aqueous), one or more organic solvents (i.e., non-aqueous), a combination or water and one or more organic solvents (i.e., semi-aqueous), and so forth. For certain ALA-based compounds, delivery enhancers, such as DMSO among others, may be used to increase the amount of ALA absorption by the local tissue, such as in the case of a mucosa layer. The delivery may also use a combination of aqueous and non-aqueous solvents. To further enhance tissue uptake, the ALA-based compound may also be incorporated into liposome or emulsions.

Another way of delivery of the ALA-based compound is through injection into the local tissue or organ.

Examples of aqueous vehicles include, for example, water, saline, dextrose or other sugar solutions, and Ringer's solution, among others. Examples of non-aqueous vehicles include, for example, fatty oils of vegetable origin (e.g., cottonseed, peanut, corn, sesame, etc.), alcohols (e.g., ethanol, isopropanol, hexadecyl alcohol, etc.), glycols (e.g., propylene glycol, polyethylene glycol, etc.), dimethylsulfoxide, ethyl lactate, acetone, tetrahydrofurfuryl alcohol, N-methyl 2-pyrrolidone, glycerol formal, Solketal, glycofurol, dimethyl isosorbide and diglyme, among others.

Optionally, additional pharmaceutically acceptable agents, for example liposome-forming agents, surfactants (e.g., where non-aqueous vehicles are used), antioxidants, physiologically compatible buffers, bacteriostats, and solutes that render the formulation isotonic with blood, among others, may be added to the formulation.

Some of the above organic solvents (e.g., dimethylsulfoxide, ethanol, etc.) may also act as tissue penetrating enhancers when included in the formulation containing.

In some embodiments, the liquid formulation containing the ALA-based compound is delivered via a vascular catheter, which is inserted into the vasculature of a subject, for instance, to a feeder artery of an organ to be examined, at which point the liquid formulation is released into the feeder artery. In this regard, various organs have distinct supply arteries, including the following among others: kidney (renal artery), liver (hepatic artery), uterus (left and right uterine arteries), ovaries and fallopian tubes (left and right ovarian arteries), and bladder (superior vesical artery), among others.

In other embodiments, the liquid formulation is applied to the surface of a tissue or organ of interest (i.e., applied topically).

For example, in some instances, a urinary catheter is inserted into the urinary tract through a cystoscope and advanced to the desired location, for example, to the urethra, bladder, ureter or kidney, at which point the liquid formulation is released. The catheter which may further be equipped with a suitable distributor (e.g., a spray head) to spray surrounding tissue, such as the bladder wall. Note that this allows one to establish higher concentrations at the bladder surface using smaller amounts ALA-based compound, as compared to certain other methods such as instillation. The cystoscope provides direct vision during the delivery of die ALA-based compound (e.g., by spray, etc.).

In some instances, a catheter is inserted, for example, either through a laparoscopic channel or directly through an incision in the abdominal wall into the peritoneal cavity, whereupon the liquid formulation is applied onto selected tissues and organs within the abdominal and/or pelvic cavities (collectively referred to as the abdominopelvic cavity or the peritoneal cavity).

In some instances, a catheter is inserted through the vagina and into the female reproductive system, whereupon die liquid formulation is applied onto selected tissues and organs.

As previously indicated, in some instances, a catheter is inserted through the working channel of an endoscope in order to apply the liquid formulation. Examples of endoscopes include cytoscopes, ureteroscopes, nephroscopes, uteroscopes, falloposcopes and laparoscopes.

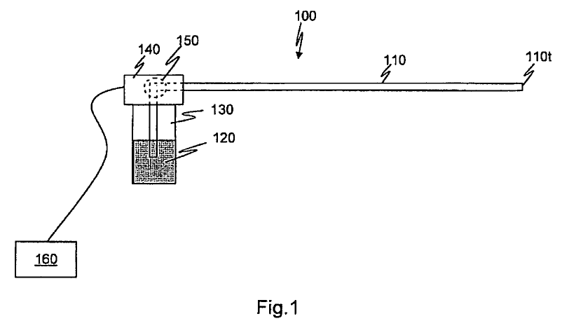

One example of a suitable device of applying liquid formulations is shown in FIG. 1, which is a schematic illustration of a delivery device 100, in accordance with an embodiment of the invention. The delivery device includes a flexible tube 110 (i.e., catheter), which may be inserted through the working channel of an endoscope. A liquid formulation 120, which in this instance contains an ALA-based compound, is located within a pressurized container 130. The device 100 is further provided with a handle 140 to control the location of the distal tip 110t of the tube 110. Tube tip 110t may be provided with a suitable distributor such as a spray head (not shown), if desired. A valve 150 may be operated to regulate the flow of the liquid formulation 120 from the pressurized reservoir 130, through the tube 110, and onto nearby tissue. The valve 140 can be operated by a suitable switch, for example, a foot controlled switch 160 or a switch located in the handle (not shown).

The container is exchangeable. It could contain the ALA-based liquid formula or powdered formula for initial diagnosis. It could also be replaced with a container which contains a liquid or powdered therapeutic agent for treatment purposes.

Thus, in a similar fashion as discussed above, a liquid formulation 120, which contains a therapeutic agent, may be located within a pressurized container 130. The therapeutic-agent-containing liquid formulation may be directly delivered to the tissue or organ after the diagnosis, for the purpose of treatment.

Specific examples of therapeutic agents for use in the invention may be selected from suitable members of the following: radioisotopes, which may be covalently bound or non-covalently bound to another species, antineoplastic/antiproliferative/anti-mitotic agents including anti-metabolites such as folic acid analogs/antagonists (e.g., methotrexate, etc.), purine analogs (e.g., 6-mercaptopurine, thioguanine, cladribine, which is a chlorinated purine nucleoside analog, etc.) and pyrimidine analogs (e.g., cytarabine, fluorouracil, etc.), alkaloids including taxanes (e.g., paclitaxel, docetaxel, etc.), alkylating agents such as alkyl sulfonates, nitrogen mustards (e.g., cyclophosphamide, ifosfamide, etc.), nitrosoureas, ethylenimines and methylmelanines, other alkyating agents (e.g., dacarbazine, etc.), antibiotics and analogs (e.g., daunorubicin, doxorubicin, idarubicin, mitomycin, bleomycins, plicamycin, etc.), antiestrogens (e.g., tamoxifen, etc.), antiandrogens (e.g., flutamide, etc.), platinum complexes (e.g., cisplatin, carboplatin, etc.), antineoplastic enzymes (e.g., asparaginase, etc.), agents affecting microtubule dynamics (e.g., vinblastine, vincristine, colchicine, Epo D, epothilone, etc.), caspase activators, proteasome inhibitors, angiogenesis inhibitors (e.g., statins such as endostatin, cerivastatin and angiostatin, squalamine, etc.), olimus family drugs (e.g., sirolimus, everolimus, tacrolimus, zotarolimus, etc.), etoposides, other agents (e.g., hydroxyurea, flavopiridol, procarbizine, mitoxantrone, campothecin, etc.), various pharmaceutically acceptable salts and derivatives (e.g., esters, etc.) of the foregoing, and combinations of the foregoing, among other agents.

Various measures may also be employed to enhance tissue uptake of the ALA-based compound, thereby reducing the time between administration of the ALA-based compound and illumination of the PPIX byproduct within the tissue.

For example, a suitable tissue penetrating enhancer (e.g., a non-aqueous organic solvent such as dimethylsulfoxide or ethanol, surfactants, etc.) may be administered to the tissue or organ immediately prior to, concurrent with, and/or subsequent to the administration of the ALA-based compound, thereby enhancing tissue uptake.

As another example, the ALA-based compound can also be delivered through local injection directly into the tissue or organ.

In other embodiments, the ALA-based compound is provided within liposomes or emulsions to enhance tissue uptake.

In still other embodiments, the liquid formulation of the ALA-based compound is locally delivered at temperatures above normal body temperature (e.g., between 38° C. and 50° C., typically about 40-44° C.) to enhance tissue uptake.

In yet other embodiments, the tissue is mechanically agitated concurrently with and/or subsequent to the delivery of the liquid formulation of the ALA-based compound to enhance uptake. For example, die tissue may be agitated by directing ultrasonic energy on the tissue to enhance uptake of the ALA-based compound.

These additional methods may be employed, for example, in order to reduce the time between administration of die ALA-based compound and illumination of the PPIX byproduct within the tissue to less than one hour, preferably to less than 30 minutes and more preferably to less than 15 minutes.

As noted above, it is well known that the metabolic formation of PPIX from ALA-based compounds preferentially occurs in neoplastic tissue. Consequently, illumination with light of a suitable wavelength (e.g., blue light) can be used to identify likely neoplastic tissue based on the distinct red fluorescence that is produced during light excitation of PPIX, a process known as photodynamic diagnosis (PDD).

Moreover, higher light doses may be used for photodynamic therapy (PDT). Without wishing to be bound by theory, it has been proposed that the mechanism for PDT involves the excitation of PPIX to a first excited state upon the absorption of a photon of light of a suitable wavelength. Energy is then transferred from the singlet state to the triplet state, which can then react with nearby oxygen molecules to create singlet oxygen, a highly reactive state of oxygen that damages lipids, nucleic acids and other cellular components, leading to cell death.

In the present invention, illumination with light of a suitable wavelength thus allows for the PPD or PPT of neoplastic tissue at or near a body cavity or body lumen surface, in particular, tissue at or near die interior of tie urinary tract (e.g., the urethra, bladder, ureters, kidneys), tissue at or near the interior of the female reproductive organs (e.g., the vagina, uterus, fallopian tubes, ovaries), and tissue at or near the interior of the abdominopelvic cavity (e.g., the female reproductive organs, including die uterus, ovaries and fallopian tubes, the liver, the gallbladder, the stomach, the small intestines, the pancreas, the spleen, the kidneys, die peritoneum, and so forth). The peritoneum is thin membrane that lines the abdominopelvic cavity and various organs therein and includes visceral peritoneum, which lies on the surfaces of the abdominal and pelvic organs (although some organs, such as die kidneys, protrude into the peritoneal cavity, but are not encased in the peritoneum), and the parietal peritoneum, which lines the inner surfaces of the walls of the abdominopelvic cavity.

To apply such light, a conventional endoscope, for example, a conventional cytoscope, ureteroscope, nephroscope, uteroscope, falloposcope, or laparoscope, may be supplied with a blue filter, or such a device may be retrofitted with a blue light source, the intensity of which depends upon whether the object is PDD or PDT. Alternatively, blue light emitting scopes are also commercially available such as the D-Light system (Karl Storz, Germany), which features a conventional white-light mode for standard viewing and a specific blue light mode for photodynamic excitation. Thus, those of ordinary skill in the art can thus construct, modify or purchase an endoscope that can readily perform the desired task. As another example, a light emitting device, for example, a bundle of optical fibers in optical communication with a light source, may be advanced down the working channel of an endoscope, in order to expose the tissue to light.

Neoplastic tissue is new and abnormal tissue, which may be malignant or benign.

Ovarian cancer is an example of malignant neoplastic tissue. Ovarian cancer commonly metastasizes by breaking through the ovarian capsule and spreading along parietal and visceral peritoneal surfaces. Ovarian cancer has a high frequency of metastasis yet generally remains localized within the peritoneal cavity. However, malignant cells can implant essentially anywhere in the peritoneal cavity. Because cancerous tissue is neoplastic, it can be diagnosed via PPD and/or treated via PPT. Other cancers that may be diagnosed and/or treated from within the peritoneal cavity include liver cancer and peritoneal mesothelioma, among others. Common cancers which may be diagnosed and/or treated from within the urinary tract include bladder cancer, ureteral cancer, uterine cancer and kidney cancer. Common cancers which may be diagnosed and/or treated from within the female reproductive system include cancer of the uterus and endometrial cancer.

Endometriosis may also be treated. Endometrial implants are examples of benign neoplastic tissue. Endometriosis is the growth of cells similar to those that form the inside of the uterus outside of the uterus. Endometrial cells are the same cells that are shed each month during menstruation. When endometrial cells grow outside the uterus, endometriosis results. These cells attach themselves to tissue outside the uterus and are called endometrial implants. The implants are most commonly found on the ovaries, the fallopian tubes, outer surfaces of die uterus or intestines, and on the surface lining of the pelvic cavity. They can also be found on the liver, vagina, old surgery scars, and even in the lung or brain. Endometrial implants are generally benign (not cancerous). However, since the tissue continues to respond to reproductive hormones, bleeding can occur from this displaced tissue during the menstrual cycle, causing the symptoms such as painful period (cramps), painful sex and pelvic pain, among others, depending on which organs are involved. Because, endometrial tissue is neoplastic, it can be diagnosed and treated via PPD or PPT. Many of the tissues and organs affected by endometrial implants may be diagnosed and/or treated from within the abdominopelvic cavity.

A laparoscope, like most endoscopes, is a thin tube-shaped instrument, which contains an illumination element such as a light source or light guide and a sensing element such as a camera. During a laparoscopy, a laparoscope is inserted through an incision in the abdominal wall to examine female pelvic organs, including the uterus, ovaries or fallopian tubes, or other organs accessible from the peritoneal cavity including the stomach, intestines, liver, gallbladder, spleen, pancreas or kidneys. In many cases laparoscopy can eliminate the need for a more extensive operation that would require a larger incision in the abdomen (laparotomy). Laparoscopy can be less risky, less stressful, and less costly than laparotomy and can often be done without requiring an overnight stay in the hospital.

In a typical laparoscopy, a surgeon first makes a small cut below the navel. A laparoscope is subsequently passed through the cut into the perineal cavity. Commonly, carbon dioxide gas is passed into the area to create a larger space to work in. This also helps the surgeon see the area better. An instrument may also be placed in the vagina to move the uterus during surgery. Moreover, additional instruments are often required, for example, in order to get a better view of certain organs or to assist with diagnostic (biopsy) or therapeutic removal of tissue. These instruments include cutting devices, lasers, electrocautery devices and biopsy devices and they can be introduced through the scope or through additional cuts in the abdominal wall.

In a laparoscopy procedure in accordance with an embodiment of the invention, the doctor first locally delivers a liquid formulation containing an ALA-based compound to a target area of a subject. As discussed above, the liquid formulation may be delivered via vascular catheter to an artery that feeds an organ in the perineal cavity. Alternatively a catheter can be inserted into the perineal cavity (e.g., through the laparoscope or a separate incision), and the liquid formulation dispensed from the catheter onto the tissue and/or organs to be examined within the abdominopelvic cavity. Moreover, one or more measures may optionally be taken to enhance tissue uptake of the ALA-based compound, for example, (a) the ALA-based compound may be provided within a liposomal formulation, (b) a suitable tissue penetrating enhancer may be administered to the tissue or organ immediately prior to, concurrent with, and/or subsequent to the administration of the ALA-based compound, (c) the liquid formulation may be applied at temperatures above normal body temperature (i.e., the formulation may be preheated to a temperature higher than 37° C.), (d) the tissue or organ may be mechanically agitated concurrently with and/or subsequent to the delivery of the liquid formulation (e.g., by inserting an ultrasound emitting instrument into the perineal cavity or vagina), or (e) a combination of two or more of the preceding measures. For example, the tissue may be agitated by advancing an ultrasound generating device down the working channel of the laparoscope, thereby directing ultrasonic energy on the tissue to enhance uptake of the ALA-based compound.

After a sufficient time has passed, the doctor may then identify neoplastic tissue via PPD (e.g., to determine whether cancer in another area of the body has spread to the tissue and organs of the peritoneal cavity or to identify abnormal tissues or organs). The doctor can also take tissue samples for biopsy based on PPD, remove abnormal tissues or entire organs (e.g., ovaries, uterus, gallbladder, or spleen) based on PPD, or conduct PPT, among other procedures.

In a typical cytoscopy procedure, an endoscope (called a cytoscope) is inserted through the urethra into the bladder. Water or saline is inserted through the cytoscope and washes the bladder. As the fluid fills the bladder, it stretches the bladder wall, enabling the physician to view the entire bladder wall. If any tissue appears abnormal, a biopsy can be performed through the cytoscope to be analyzed.

In a cytoscopy procedure in accordance with an embodiment of the invention, the doctor first locally delivers a liquid formulation containing the ALA-based compound to the target surface of the subject. For example, a catheter can be inserted into the bladder, either directly or via a working channel of a cytoscope, and the bladder filled with the liquid formulation in order to expose the entire bladder wall. One or more measures such as those described above may optionally be taken to enhance tissue uptake of the ALA-based compound. For example, the doctor may also (a) wash the bladder with a penetration solution or with a washing solution such as saline (which may initially flow continuously through the catheter, but is stopped at the time of diagnosis and/or treatment), (b) empty the bladder and (c) fill or wash the bladder with liquid formulation containing the ALA-based compound (e.g., a relatively small amount, for instance, about 1-200 mL may be applied—in some embodiments the formulation is circulated inside the bladder to increase the interaction time and contacting area to further increase tissue uptake and improve biofunction). The doctor may also inject the ALA-based compound solution into the arteries of the bladder for diagnosis purposes. After a sufficient time has passed, the doctor can then identify neoplastic tissue via PPD, and also conduct treatment, if desired. The doctor can also take tissue samples for biopsy or treatment based on PPD, or conduct PPT, among other procedures. For example, light suitable for this purpose may be provided using a light-emitting cytoscope or by inserting a suitable light source through a working channel of die cytoscope.

In a typical ureteroscopy procedure, an endoscope (called a ureteroscope) is inserted through the urethra and bladder and into the ureter.

In a ureteroscopy procedure in accordance with an embodiment of the invention, the doctor delivers a liquid formulation containing an ALA-based compound to the ureter (or into the kidney) via a ureteral catheter or via the working channel of the ureteroscope. One or more measures such as those described above may be taken to enhance tissue uptake of the ALA-based compound. After a sufficient time has passed, the doctor can then identify neoplastic tissue via PPD. The doctor can also take tissue samples for biopsy or treatment based on PPD, or conduct PPT, among other procedures. For example, light suitable for this purpose may be provided using a light-emitting ureteroscope or by inserting a suitable light source through the working channel of die cytoscope.

In a typical ureteroscopy procedure, an endoscope (called a uteroscope) is inserted through the cervix and into the uterus to examine the uterus. Similarly, in a known falloposcopy procedure, a tiny flexible fallopian catheter is inserted through the cervical canal and uterine cavity into the fallopian tube, whereupon an even smaller flexible fiber optic endoscope, called a falloposcope, is threaded through the catheter into the fallopian tube.

In an embodiment of the present invention, a liquid formulation containing die ALA-based compound is delivered to the uterus and/or fallopian tube via a catheter that is inserted through the cervix One or more measures such as those described above may be taken to enhance tissue uptake of the ALA-based compound. After a sufficient time has passed, the doctor can then identify neoplastic tissue via PPD (and treat neoplastic tissue via PPT) using a suitable light-emitting uteroscope or falloposcope. The doctor may also take tissue samples for biopsy or treatment based on PPD. Alternatively, the doctor can use laparoscopy to identify neoplastic tissue in the uterus or fallopian tube via PPD, to take tissue samples for biopsy or treatment based on PPD, and conduct PPT.

The table below lists various organs and tissue, modes by which the ALA-based compound can be locally delivered to the tissue/organ, and modes by which the tissue/organ can be illuminated of PPD, after delivery of the ALA-based compound.

| Organ/Tissue | Delivery Mode | Illumination Mode |
|---|---|---|
| Bladder | Topical spray (ALA-compound | From within (e.g., cytoscope) |
| | could be liquid or powder) | |
| | Topical from filled bladder | |
| | Feeder artery through injection | |
| Ureters, | Topical from within | From within (e.g., ureteroscope) |
| Kidney | Topical from peritoneal cavity | From peritoneal cavity (e.g., laparoscope) |
| Vagina | Topical from within | From within |
| | Feeder artery injection | |
| Uterus, | Topical from within | From within (e.g., uteroscope, falloposcope) |
| Fallopian tubes | Topical from peritoneal cavity | From peritoneal cavity (laparoscope) |
| | Feeder artery injection | |
| Ovary | Topical from peritoneal cavity | From peritoneal cavity (laparoscope) |
| | Feeder artery injection | |
| Peritoneum | Topical from peritoneal cavity | From peritoneal cavity (laparoscope) |
| Liver | Topical from peritoneal cavity | From peritoneal cavity (laparoscope) |
| | Feeder artery injection | |
| Stomach, | Topical from peritoneal cavity | From peritoneal cavity (laparoscope) |
| Intestines, | | |
| Gallbladder, | | |
| Pancreas, | | |
| Spleen | | |
| Colon | Topical spray | From within (e.g., colonoscope) |
| | Topical from filled colon | |
| | Feeder artery injection | |

Various aspects of the invention of the invention relating to the above are enumerated in the following paragraphs:

Aspect 1. A method of diagnosis or treatment, comprising: (a) locally delivering a 5-aminolevulinic-acid-based compound (ALA-based compound) to a tissue or organ within a subject's body; and (b) illuminating the tissue or organ with light having a wavelength suitable to induce fluorescence of protoporphyrin IX (PPIX).

Aspect 2. The method of Aspect 1, wherein the method is used to visually identify areas of the tissue or organ that have increased PPIX levels.

Aspect 3. The method of Aspect 1, wherein the method is used to photodynamically treat areas of the tissue or organ that have increased PPIX levels.

Aspect 4. The method of Aspect 1, wherein the ALA-based compound is 5-aminolevulinic acid or a salt thereof.

Aspect 5. The method of Aspect 1, wherein the ALA-based compound is a 5-aminolevulinic acid ester or a salt thereof.

Aspect 6. The method of Aspect 1, wherein the ALA-based compound is a 5-aminolevulinic acid thioester or a salt thereof.

Aspect 7. The method of Aspect 1, wherein the ALA-based compound is locally delivered to a feeder artery for the tissue or organ.

Aspect 8. The method of Aspect 1, wherein the ALA-based compound is topically applied to a surface of the tissue or organ.

Aspect 9. The method of Aspect 1, wherein the ALA-based compound is locally delivered as a liquid formulation.

Aspect 10. The method of Aspect 9, wherein the liquid formulation comprises a tissue penetrating enhancer.

Aspect 11. The method of Aspect 10, wherein the tissue penetrating enhancer is an organic solvent.

Aspect 12. The method of Aspect 9, wherein the liquid formulation comprises liposomes that contain the ALA-based compound.

Aspect 13. The method of Aspect 9, wherein the liquid formulation is delivered by a device that comprises a catheter.

Aspect 14. The method of Aspect 9, wherein the liquid formulation is delivered at a temperature of 40° C. or more.

Aspect 15. The method of Aspect 1, wherein the ALA-based compound is delivered while mechanically agitating the tissue or organ.

Aspect 16. The method of Aspect 15, wherein the tissue or organ is mechanically agitated with ultrasonic energy.

Aspect 17. The method of Aspect 1, wherein the tissue or organ is illuminated within 30 minutes after local delivery of the ALA-based compound.

Aspect 18. The method of Aspect 1, wherein the fluorescence is viewed from within the body using an endoscope.

Aspect 19. The method of Aspect 18, wherein the ALA-based compound is locally delivered as a liquid formulation through a catheter that is inserted through a working channel of the endoscope.

Aspect 20. The method of Aspect 1, wherein the fluorescence is viewed from within the urinary tract using a cytoscope, ureteroscope or nephroscope.

Aspect 21. The method of Aspect 1, wherein the fluorescence is viewed within the female reproductive organs using a uteroscope or falloposcope.

Aspect 22. The method of Aspect 1, wherein the fluorescence is viewed within the abdominopelvic cavity using a laparoscope.

Aspect 23. A method of diagnosis or treatment, comprising: (a) locally delivering a 5-aminolevulinic-acid-based compound (ALA-based compound) to a tissue or organ within a subject's body; (b) illuminating the tissue or organ with light having a wavelength suitable to induce fluorescence of protoporphyrin IX (PPIX); and (c) locally delivering a therapeutic agent to the tissue or organ for the purpose of treatment.

Although various embodiments are specifically illustrated and described herein, it will be appreciated that modifications and variations of die present invention are covered by the above teachings and are within the purview of any appended claims without departing from the spirit and intended scope of the invention.

1. A method of diagnosis or treatment, comprising:
(a) locally delivering a 5-aminolevulinic-acid-based compound (ALA-based compound) to a tissue or organ within a subject's body; and
(b) illuminating the tissue or organ with light having a wavelength suitable to induce fluorescence of protoporphyrin IX (PPIX).

2. The method of claim 1, wherein said method is used to visually identify areas of the tissue or organ that have increased PPIX levels.

3. The method of claim 1, wherein said method is used to photodynamically treat areas of the tissue or organ that have increased PPIX levels.

4. The method of claim 1, wherein said ALA-based compound is 5-aminolevulinic acid or a salt thereof.

5. The method of claim 1, wherein said ALA-based compound is a 5-aminolevulinic acid ester or a salt thereof.

6. The method of claim 1, wherein said ALA-based compound is a 5-aminolevulinic acid thioester or a salt thereof.

7. The method of claim 1, wherein said ALA-based compound is locally delivered to a feeder artery for the tissue or organ.

8. The method of claim 1, wherein said ALA-based compound is topically applied to a surface of the tissue or organ.

9. The method of claim 1, wherein said ALA-based compound is locally delivered as a liquid formulation.

10. The method of claim 9, wherein said liquid formulation comprises a tissue penetrating enhancer.

11. The method of claim 10, wherein said tissue penetrating enhancer is an organic solvent.

12. The method of claim 9, wherein said liquid formulation comprises liposomes that contain said ALA-based compound.

13. The method of claim 9, wherein said liquid formulation is delivered by a device that comprises a catheter.

14. The method of claim 9, wherein said liquid formulation is delivered at a temperature of 40° C. or more.

15. The method of claim 1, wherein said ALA-based compound is delivered while mechanically agitating the tissue or organ.

16. The method of claim 15, wherein said tissue or organ is mechanically agitated with ultrasonic energy.

17. The method of claim 1, wherein said tissue or organ is illuminated within 30 minutes after local delivery of said ALA-based compound.

18. The method of claim 1, wherein said fluorescence is viewed from within the body using an endoscope.

19. The method of claim 18, wherein said ALA-based compound is locally delivered as a liquid formulation through a catheter that is inserted through a working channel of said endoscope.

20. The method of claim 1, wherein said fluorescence is viewed from within the urinary tract using a cytoscope, ureteroscope or nephroscope.

21. The method of claim 1, wherein said fluorescence is viewed within the female reproductive organs using a uteroscope or falloposcope.

22. The method of claim 1, wherein said fluorescence is viewed within the abdominopelvic cavity using a laparoscope.

23. A method of diagnosis or treatment comprising:
(a) locally delivering a 5-aminolevulinic-acid-based compound (ALA-based compound) to a tissue or organ within a subject's body;
(b) illuminating the tissue or organ with light having a wavelength suitable to induce fluorescence of protoporphyrin IX (PPIX); and
(c) locally delivering a therapeutic agent to the tissue or organ for the purpose of treatment.
